(19) United States
(12) Patent Application Publication
(10) Pub. No.: US 2005/0037100 A1
(43) Pub. Date: Feb. 17, 2005

(54) Utilization of extracts from iris plants, cimicifuga racemose and tectorigenin as an estrogen-like organ-selective medicament without uterotropic

(75) Inventors: Wolfgang Wuttke, Bovenden (DE); Hubertus Jarry, Neu-Eichenberg (DE); Volker Christoffel, Neumarkt (DE); Barbara Spengler, Neumarkt (DE); Michael Popp, Lauf/Pegnitz (DE)

Correspondence Address: CHRISTIE, PARKER & HALE, LLP, PO BOX 7068, PASADENA, CA, 91109-7068, US

(73) Assignee: Bionorica AG

(21) Appl. No.: 10/951,583

(22) Filed: Sep. 27, 2004

Related U.S. Application Data
(63) Continuation of application No. 09/646,740, filed on Sep. 18, 2000, now abandoned.

(30) Foreign Application Priority Data
Mar. 19, 1998 (DE) 19812204.7

(51) Int. Cl. A61K 35/78

(52) U.S. Cl. 424/764; 424/773; 514/25

(57) ABSTRACT

The present invention relates to the use of extracts from Iridaceae and from Cimicifuga racemosa, and of tectorigenin as an estrogen-type, organoselective medicament for the selective treatment and/or prophylaxis of cardiovascular diseases, in particular atherosclerose, osteoporose and climacteric disorders, e.g. for preventing or alleviating hot flushes. Uterotrophic effects are practically not observed.

The present invention relates to the use of extracts from Iridaceae in accordance with claim 1, and those from Cimicifuga racemosa as an estrogen-type, organoselective medicament, and tectorigenin and/or tectorigenin glycosides as a medicament in accordance with claim 8.

17-β-estradiol, which is formed in the ovaries (whenever estradiol is mentioned hereinafter, this always refers to physiological 17-β-estradiol) [hereinafter also referred to as E₂], generally has a proliferation-promoting effect in the organism. Apart from controlling the female cycle, it i.a. has a homeostatic influence on the metabolism of the bone and prevents the formation of atherotic plaques at the endothelium of the vessels.

During menopause, lowering of the estradiol level takes place due to cessation of the ovarial function. This results in a weakening of proliferative processes, and in the hypothalamus results in an intensified activity of the GnRH impulse generator. (The gonadotropin-releasing hormone impulse generator is a timer in the hypothalamus, as it were, and times the pulsatile LH secretion, with steroids influencing amplitude and frequency.) In climacteric women, the resulting, stimulated LH secretion brings about the so-called “hot flushes” which are felt to be disturbing.

In the absence of sufficiently high estradiol levels in the blood, osteoclast activity and thus destruction of the bone mass is predominant, accompanied by an increased risk of skeleton breakage. At the same time, there is in the long term a risk of plaque formation in the vascular system and thus an increased risk of infarctions.

Extracts from Cimicifuga racemosa and from Belamcanda sinensis are both known from popular medicine to be capable of alleviating peri-menopausal and post-menopausal disorders. Hitherto this has been explained through the fact that the extracts of both plant drugs exhibit an estrogen-type effect with all the positive effects thereof on a multiplicity of organs of the human body, particularly the brain, ovaries, bones, vascular system. Estrogen-type effects on uterus, vagina, breast tissue and liver would in turn be disadvantagous. What is undesirable, however, is that up to the present, a medicament from these plant drugs which might be used for organoselective prophylaxis or therapy in cases of estrogen deficiency, has not been available in the prior art.

Starting out from this state of the art, it is therefore object of the present invention to furnish plant medicaments with an estrogen-type effect, the effect of which is organoselective with no effect or only a slight effect on the uterus.

This object is independently achieved through the features of claim 1 with respect to the use of extracts from Iridaceae, through the use of extracts from Cimicifuga racemosa in accordance with claim 3. The above object is moreover achieved by the features of claim 2 with respect to a medicament on the basis of tectorigenin and/or its glycosides in accordance with claim 8.

Another independent solution is represented by a plant extract containing tectorigenin and/or tectorigenin glycoside or enriched with tectorigenin and/or tectorigenin glycoside, in accordance with claim 11.

Both in in-vitro and in-vivo experiments it was surprisingly found that extracts produced both from Iridaceae, particularly Belamcanda sinensis, and from Cimicifuga racemosa with organic solvents or with supercritical CO₂ organoselectively act on the central nervous system, the bone system and the vascular system, with an effect on the uterus—the so-called uterotrophic effect—not existing. The extracts used in accordance with the invention are thus suited for producing a ready-formulated medicament for the selective treatment and/or prophylaxis of osteoporosis.

They are moreover suited for production of a ready-formulated medicament for the selective treatment and/or prophylaxis of cardiovascular diseases, particularly of atherosclerosis.

They are moreover suited for producing a ready-formulated medicament for the selective treatment and/or prophylaxis of peri-menopausal and post-menopausal psychovegetative disorders such as, e.g., hot flushes.

It was moreover found that the component tectorigenin, which was isolated from Belamcanda sinensis, essentially exerts the same effects as the whole extract.

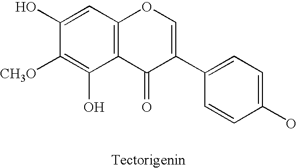

This component is also found, besides Belamcanda sinensis, in other Iridaceae such as, e.g., Iris germanica, I. tectorum, I. illyrica, I. dichotoma.

Taxonomically speaking, Belamcanda sinensis is classified as follows:

| | |
|---|---|
| Order | Liliales |
| Family | Iridaceae |
| Genus | Belamcanda |
| Species | Belamcanda sinensis (Leman) DC. = Pardanthus |
| | chinensis (L.) Ker-Gawler, also: Ixia chinensis L. |
| | (=Gemmingia chinensis (L.) 0. Kuntze) |

Preferably rhizomes, stalks, leaves and/or petals of the plants are used for producing the extracts.

A fundamental phytochemical description of Belamcanda sinensis and its components was given in the dissertation by Ms. A. Nenninger: (LMU MU nchen, 1997) entitled: “Phytochemische und pharmakologische Untersuchungen von Belamcanda sinensis, einer Arzneipflanze der TCM und anderer Irisarten”.

With the medicaments of the invention, medicaments from Cimicifuga racemosa and Belamcanda sinensis and other Iridaceae and tectorigenin-based medicaments are for the first time available, which act as full estrogen receptor agonists in bones, in the cardiovascular system and in the brain.

Further advantages and features of the present invention become clear from the description of experimental data and by referring to the drawings, showing:

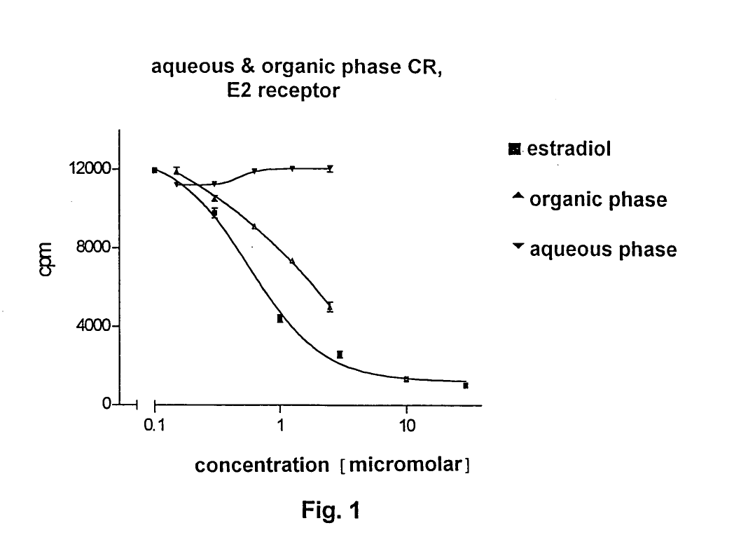

FIG. 1: a comparison of the organic and aqueous phases of Cimicifuga racemosa. Displacement graph of a representative estrogen receptor—ligand binding assay. The concentration of the start solution is 17.66 mg/ml, followed by dilutions 1:2, 1:4 etc. up to 1:64;

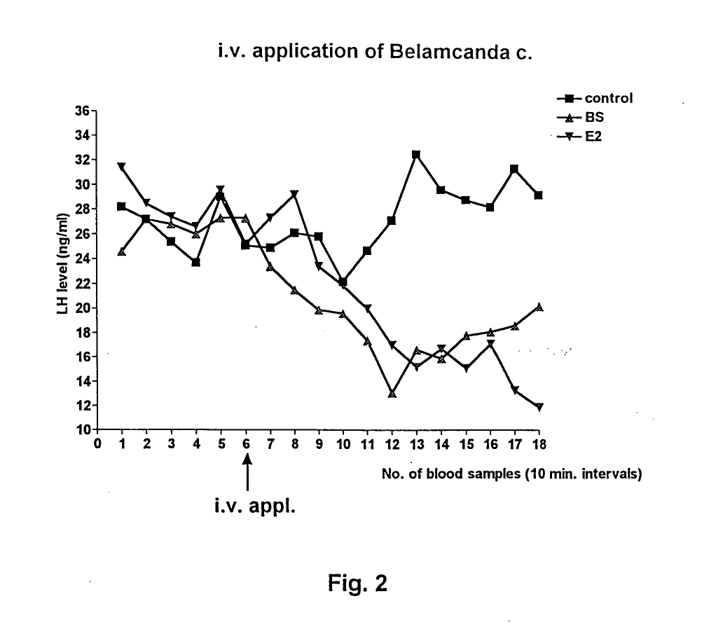

FIG. 2: serum LH prior to, and 2 hours after, intravenous injection of Belamcanda sinensis extract, E2 and vehicle. The Belamcanda sinensis extract has a similar capacity of lowering the elevated Serum LH levels as E2;

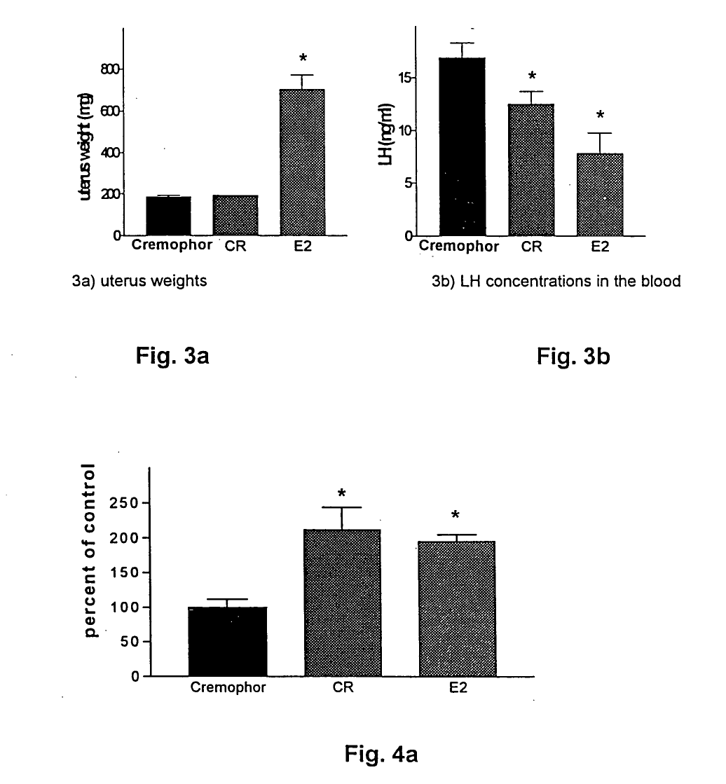

FIG. 3. effects of Cimicifuga racemosa and E2 on uterus weights (FIG. 3a) and LH levels in the blood (FIG. 3b) in ovariectomised rats after seven-day subcutaneous treatment; (mean values+SEM, n=8,*=p<0.05 vs. cremophor as vehicle);

FIG. 3a) uterus weights;

FIG. 3b) LH concentrations in the blood;

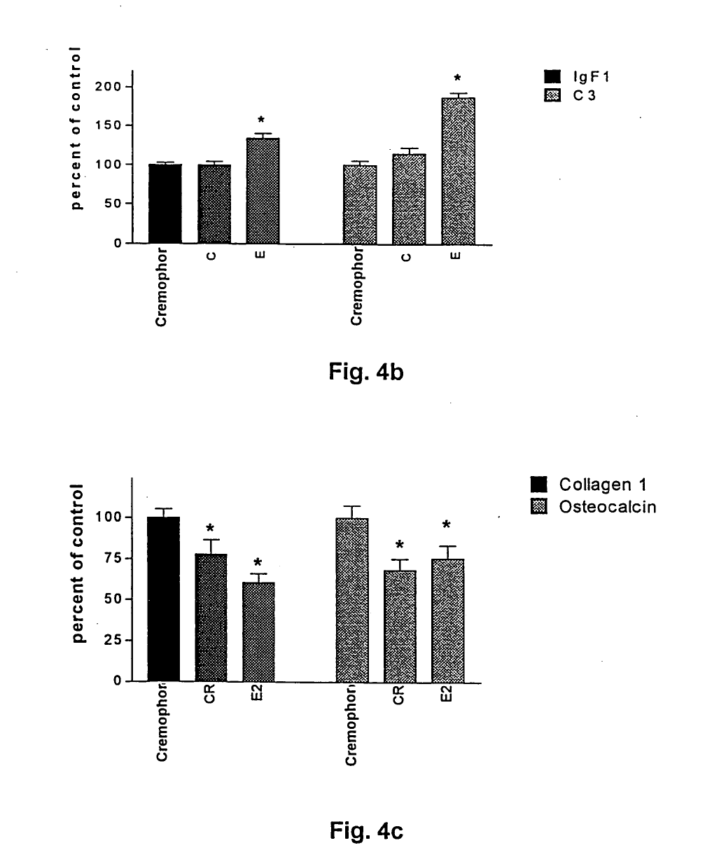

FIG. 4a) effects of Cimicifuga racemosa and E2 in ovariectomised rats after seven-day subcutaneous treatment; (mean values+SEM, n=8,*=p<0.05 vs. cremophor as vehicle) on the expression of the mRNA for E2-receptor a in the preoptic region of the hypothalamus;

FIG. 4b) the expression of the mRNA for IGF1 and C3 in the uterus of ovariectomised rats after 7 days of subcutaneous administration; and

FIG. 4c) the expression of the mRNA for collagen 1 (Coll1) and osteocalcin in the bone of ovariectomised rats after 7 days of subcutaneous administration.

### EXPERIMENTAL EVIDENCE FOR THE ESTROGENIC EFFECT OF Cimicifuga racemosa AND Belamcanda sinensis

Selective estrogenic effect was demonstrated in stages in the course of a series of test systems of various degrees of complexity.

1. In-Vitro Experimentation

1.1 In-Vitro Experiments for Cimicifuga racemosa

Recognition of the estrogen-type structure of components by an antibody directed against 17-β-estradiol (=E2) was shown in vitro.

The Cimicifuga racemosa extract was evaporated over residue. By phase distribution between dichloromethane and water, substances having different polarities were enriched. Binding affinities of the components of both phases were determined in vitro on estrogen receptors from pig's uterus. The cytosolic estrogen receptors from the pig uteri were isolated in accordance with standard procedures and used for the ligand displacement experiments.

Herein it was found that the estrogen-type structures e.g. from Cimicifuga racemosa are not hydrophilic in nature but lipophilic inasmuch as they may be extracted from the extract by means of an organic solvent. The substances present in the organically extracted phase bind about ten times more strongly to the antibody than the substances remaining in the aqueous phase.

The difference between the two phases is even greater in the estradiol receptor binding assay. The similarity of the binding substance with estradiol must be high enough to enable a selective—competitive—interaction with the estradiol receptor to take place in a cell-free preparation. Inside this test system, the aqueous phase does not possess any activity, whereas the organic phase binds very strongly to the receptor.

The results are shown in FIG. 1.

1.2 In-Vitro Belamcanda sinensis

It is known from other studies that extracts from Belamcanda sinensis also possess components which are recognised by an antibody against 17-β-estradiol and bind to the 17-β-estradiol receptor (cf. Nenninger loc.cit.). Surprisingly, however, the inventors of the present application have found that these extracts have different estrogenic effects on different organ systems, particularly that they do not have a uterotrophic effect.

2. In-Vivo Experiments: Evidence for the Estrogenic Effect on Ovariectomised Rat

Binding to the receptor E2 is very selective; it is, however, not possible to say whether the subsequent processes within the cell are promoted or inhibited, i.e. whether the substance is an agonist or an antagonist. This property can only be determined in suitable cellular systems or in the overall animal.

The ovariectomised rat is a recognised model for the post-menopausal woman in whom the endogenous estradiol production has subsided. As a result of the external supply of 17-β-estradiol or of substances which have an estrogen-type effect, there occurs a restauration of estrogen-sensitive anatomical-morphological parameters, such as increase of the uterus weights and the occurrence of hornified cells, i.e. plaque epithelium cells at the vaginal epithelium, or hormonal changes such as lowering of the LH levels in the blood of the treated animals.

All experiments described hereinbelow were carried out with ovariectomised Sprague-Dawley rats (═Ovx rats) having a weight between 240 and 280 g.

2.1 Single Administration of Belamcanda sinensis

The onset of the effect of the estradiol-type effect of Belamcanda sinensis extract occurs very quickly. Even after a single i.v. administration of vehicle, estradiol and Belamcanda sinensis extract to ovx rats, pulsatility ceases both under E2 and under Belamcanda sinensis. In the medicament value development, there result significant inhibitions of the serum LH levels, both in comparison with the previous values and in comparison with the cremophor-treated control animals. Cremophor is an emulsifier on the basis of polyethoxylated castor oil derivatives.

The results are represented in FIG. 2.

In the uterus of the animals six hours after injection of the Belamcanda sinensis extract, the expression of the uterine VEGF, IGF1 and C3 genes is not changed in comparison with the controls, whereas the estradiol injection brings about a clear increase of the gene expression of these three estrogen-regulated proteins. The constitutively expressed CCO gene was not significantly influenced by any one of these treatments.

These findings indicate that components of Belamcanda sinensis bring about an inhibition of the GnRH pulse generator in hypothalamic estrogen-receptive structures and thus have estrogen-agonistic effects. Hereby the hypophysary LH secretion is inhibited significantly both by components in Belamcanda sinensis and by estradiol. In contrast with estradiol, the components in Belamcanda sinensis do not have a uterotrophic effect. Estradiol significantly regulates the gene expression of VEGF, IGF1 and C3 upwardly, an effect which is not observed under Belamcanda sinensis.

Execution of the acute experiment on the effect of an i.v. injection of Belamcanda sinensis extract

24 rats (i.e. 8 animals/group) had a jugular vein catheter implanted under ether anesthesia on the day preceding the experiment. On the day of the experiment, 6 blood samples were taken at 10-min intervals. Immediately following taking of the 6th sample, 62.5 mg of the Belamcanda sinensis extract or 10 μg 17-β-estradiol (E2) or the solvent (5%) cremophor in isotonic NaCl 1 ml), respectively, were injected intravenously, and blood samples were taken for another 2 hours in 10-minute intervals. 6 hours after the intravenous administration, the animals were decapitated, blood was obtained and the uteri removed, weighed and deep-frozen in liquid nitrogen.

2.2 One-Time Administration of Tectorigenin

Following a single administration of tectorigenin, the time development of influence on the LH levels in the blood and the estradiol-type immunoreactivity were determined. The concentration of tectorigenin in the blood of the animals, determined with the aid of E2-RIA, after 20 min corresponds to about 100 pg equivalent estradiol.

Tectorigenin triggers a rapid LH reduction. The kinetics of the LH reduction achieved under tectorigenin up to the time 60 min following i.v. administration precisely correspond to the one of estradiol, but then do not result in further reduction but slowly increases again.

Execution: OVX rats had catheters placed in the vena jugularis externa under ether anesthesia 24 hours before the beginning of the experiment, in accordance with the method of Harms and Ojeda (Harms PG; Ojeda SR: A rapid and simple procedure for chronic cannulation of the rat jugular vein. J. Appl. Physiol. (1974) 36: 391-392). The tube end was positioned in a skin pocket in the neck. In order not to have to touch the animals for obtaining the blood samples, the catheter was prolonged with the aid of a silicone tube. Catheter and tube were rinsed with Ringer solution containing 50 IU heparin/ml.

Blood samples of 100 μl each were drawn from the animals at 10-min intervals, and the withdrawn volume replaced with Ringer/heparin solution. After the 6th sample, 1.0 ml of the respective test solution was applied intravenously. As test solutions there were used: 2% cremophor (=vehicle solution), tectorigenin 7 mg/ml vehicle, 17-β-estradiol 10 μg/ml vehicle. Blood was taken at ten-minute intervals through additional 140 min.

The blood samples thus obtained were filled into a 0.5 ml Eppendorf reaction vessel containing 10 μl heparin-Losung (5000 IU/ml, Liquemin), centrifuged for 10 min at 10 000*g, and the plasma stored at −20° C. until performance of the radioimmunoassays.

The RIAs for LH and Prolaktin are based on antisera, reference and iodisation preparations from NIH (Bethesda, Md., USA). The concentrations of estradiol and of the cross-reactive isoflavones were measured with the aid of an RIA from DPC, Bad Nauheim.

2.3 Effect of Belamcanda sinensis Extract After Administration Through 7 Days

The effects of repeated administration of estradiol, Belamcanda sinensis extract and vehicle on overall weight, uterus weight, hormone level and gene activation of uterus and bone were examined on ovariectomised rats after daily s.c. application through seven days.

The average body weights of the cremophor- and Belamcanda sinensis-treated animals do not differ, whereas the E₂-treated animals were significantly lighter. Neither do the uterus weights of the animals treated with cremophor and Belameanda sinensis differ significantly, whereas the E2-treatment more than tripled the uterus weights.

The serum LH levels in the Belamcanda sinensis-treated animals were reduced slightly, but significantly in comparison with the cremophor controls; reduction through estradiol was more marked.

In the uterine mRNA extract, estradiol significantly raised the gene expression of VEGF to 149% of the control value after a one-week treatment. Unter Belamcanda sinensis extract, expression was raised slightly but not significantly. Expression of the non estrogen-regulated constitutive genes for the cytochrome C oxidase (═CCO) was not influenced.

In extracts of the femur head, the collagen-1A1, osteocalcin, IGF1 and TGFβ-mRNA expression was determined. Estradiol as well as Belamcanda sinensis significantly inhibited the expression of all 4 genes without having an influence on the constitutive CCO gene.

The different effects of estradiol and Belamcanda become very clear after the seven-day treatment. Belamcanda sinensis extract has an estradiol-agonistic influence on the hypophysary LH secretion by inhibiting the GnRH impulse generator, and on the gene expression of four estrogen-regulated genes in the bone. In contrast, there is no estrogenic effect on the uterus: neither the uterus weight nor the estrogen-regulated VEGF gene are influenced by the Belamcanda sinensis extract. In contrast, estradiol brings about ballooning of the uterus and an activation of the VEGF gene.

Execution of the subacute test on the effect of daily s.c. injection through 7 days:

8 animals each per test group (24 altogether) were daily injected subcutaneously between 8:00 and 9:00 a.m. with 62.5 mg Belamcanda sinensis extract and 10 μg estradiol or the solvent (cremophor 5%, 1 ml), respectively. 6 h after the last application, the animals were decapitated and from every animal the aorta, the uterus and the left femur head were removed, cleaned, and frozen in liquid nitrogen.

In the blood samples, LH and the estradiol immunoreactivity were determined.

2.4 Repeated Administration of Cimicifuga racemosa

14 days following ovarectomy at the earliest, the animals have the respective test substance injected subcutaneously in a dose of 62.5 mg Cimicifuga racemosa/rat or 8 μg estradiol/rat once daily in the morning over a period of 7 days. Both substances were dissolved in 5% cremophor, the control animals only received the vehicle.

Following decapitation of the animals, brains, uterus and femur were prepared for mRNA-recovery. The LH concentration in the blood of the animals was determined by means of RIA. The expression of the estrogen-regulated genes in the above identified organs was determined by means of semi-quantitative RT-PCR.

The uteri of the estradiol-treated animals have more than three times the weight of those of the animals treated with Cimicifuga racemosa and vehicle which basically do not differ in their mean values. This means that the components of Cimicifuga racemosa have no influence on the uterus of the animals. This is also true for the vagina, where no hornification of the epithelium tissue occurs in the animals treated with Cimicifuga racemosa and vehicle, quite contrary to the estradiol-treated animals.

The LH levels of the vehicle-treated animals remain high, however are lowered significantly both by estradiol and Cimicifuga racemosa.

The results are shown in FIGS. 3a) and 3b).

| | | |
|---|---|---|
| Cremophor | Cimicifuga | |
| [control] | racemosa | E2 |

Uterus weights (wet)

| | | | |
|---|---|---|---|
| Number animals | 8 | 8 | 8 |
| Mean values [mg] | 185.6 | 192.3 | 702.1 |
| SD | 18.81 | 22.53 | 194.97 |
| SEM | 6.65 | 7.97 | 68.92 |

LH concentrations in the blood

| | | | |
|---|---|---|---|
| Number animals | 8 | 8 | 8 |
| Mean values [ng/ml] | 16.9 | 12.5 | 7.83 |
| SD | 3.99 | 3.4 | 5.57 |
| SEM | 1.41 | 1.2 | 1.97 |

As another marker for the estrogene effect, the activation of mRNA of estrogen-induceable proteines was measured. What was measured here was tissue from uterus, from bone tissue (femur) and from the preoptic region of the hypothalamus.

In the hypothalamus, both Cimicifuga racemosa and E2 stimulate the expression of the mRNA for the estrogen receptor α (FIG. 4a). In the bone tissue, too, Cimicifuga racemosa behaves like an estrogen and reduces, in analogy with estradiol, the expression of the mRNA for the bone-specific collagen 1 and for osteocalcin genes (FIG. 4b).

In contrast, no effect of Cimicifuga racemosa on estrogen-regulated genes in the uterus is observed. Only estradiol increases the mRNA for IGF1 and complement factor C3 (FIG. 4c).

These findings prove that the components from Cimicifuga racemosa selectively act on single organs: the extract acts estrogenically in the hypothalamus (expression of the E2 receptor a, liberation of LH) and on the bone, proven by the expression of the genes for collagen 1 and osteocalcin. Other than estradiol, however, Cimicifuga racemosa does not have an effect on the uterus, as the absence of an effect on the uterus weights and the expression of the genes for IGF1 and C3 shows.

By the experiments carried out in vitro and in vivo, it could be demonstrated that Cimicifuga racemosa and Belamcanda sinensis extracts exert an estrogenic effect. Surprisingly it was found that the extracts from the named drugs act organoselectively on central nervous system, bone and vessels, but not on the uterus, and are thus excellently suited for the prophylaxis and therapy of estrogen deficiency without having a negative influence on the endometrium.

Identical effects are achieved by the tectorigenin contained in Belamcanda.

Thus for the first time medicaments having an estrogen-type effect, however without a uterotrophic effect, are available.

The like medicaments may be used for the treatment and/or prophylaxis of cardiovascular diseases, particularly atherosclerosis, osteoporosis, and of peri- and post-menopausal psychovegetative disorders such as, e.g., hot flushes.

Among types of application, oral, intravenous and subcutaneous application are prominent.

1. Use of extracts from Iridaceae or producing an estrogen-type, organoselective medicament having no uterotrophic effect or one that is at least negligible, under the proviso that Belamcanda chinesis extract is not used if the medicament is used for alleviating peri-menopausal and post-menopausal disorders.

2-9. (Cancelled)

10. A method for producing an estrogen-type effect in a patient without causing a substantial uterotrophic effect, the method comprising administering to the patient an effective amount of a medicament comprising an extract from an Iridaceae plant, wherein the plant is selected from the group consisting of Belamcanda chinensis, Iris Germanica, Iris tectorum, Iris Illyrica, and Iris dichotoma, and at least one of tectorigenin and tectorigenin glycoside; with the proviso that the medicament does not comprise Belamcandra chinensis extract if the medicament is used for treating a peri-menopausal or post-menopausal disorder.

11. A method according to claim 10, wherein the plant is a Belamcandra chinensis.

12. A method according to claim 10, wherein the extract comprises at least one of tectorigenin and a tectorigenin glycoside.

13. A method according to claim 10, wherein the extract is enriched with at least one of tectorigenin and tectorigenin glycoside.

14. A method according to claim 10, wherein the production of the estrogen-type effect comprises treatment and/or prophylaxis of a cardiovascular disease.

15. A method according to claim 14, wherein the cardiovascular disease is atherosclerosis.

16. A method according to claim 10, wherein the production of the estrogen-type effect comprises treatment and/or prophylaxis of osteoporosis.

17. A method according to claim 10, wherein the production of the estrogen-type effect comprises treatment and/or prophylaxis of a climacteric disorder.

18. A method according to claim 10, wherein the production of the estrogen-type effect comprises preventing or alleviating hot flushes.

19. A method according to claim 10, wherein the extract is produced by means of an organic solvent or supercritical CO₂.

20. A method for producing an estrogen-type effect in a patient without causing a substantial uterotrophic effect, the method comprising administering to the patient an effective amount of a medicament comprising an extract from an Iridaceae plant, wherein the plant is selected from the group consisting of Belamcanda chinensis, Iris Germanica, Iris tectorum, Iris Illyrica, and Iris dichotoma, with the proviso that the medicament does not comprise Belamcandra chinensis extract if the medicament is used for treating a peri-menopausal or post-menopausal disorder.

21. A method for producing an estrogen-type effect in a patient without causing a substantial uterotrophic effect, the method comprising administering to the patient an effective amount of a medicament comprising at least one of tectorigenin and tectorigenin glycoside.
